(19) United States
(12) Patent Application Publication
(10) Pub. No.: US 2025/0387538 A1
(43) Pub. Date: Dec. 25, 2025

(54) OSTEOGENIC COMPOSITIONS AND METHODS OF MAKING THE SAME

(71) Applicant: Alphatec Spine, Inc., Carlsbad, CA (US)

(72) Inventor: Sahil Jalota, Carlsbad, CA (US)

(21) Appl. No.: 19/070,083

(22) Filed: Mar. 4, 2025

Related U.S. Application Data
(62) Division of application No. 18/748,469, filed on Jun. 20, 2024.

(51) Int. Cl. A61L 27/36; A61L 27/22; A61L 27/38

(52) U.S. Cl. CPC A61L 27/365; A61L 27/227; A61L 27/3608; A61L 27/3687; A61L 27/3691; A61L 27/3821; A61L 27/3847; A61L 2300/414; A61L 2430/40

(57) ABSTRACT

An osteogenic composition comprises growth-factor augmented cortical bone and cancellous bone having viable endogenous osteogenic cells that remain adherent to the cancellous bone. The growth-factor augmented cortical bone comprises demineralized cortical bone amplified with growth factors derived from a demineralized bone, and the cancellous bone not having been subjected to a loosening agent. The osteogenic composition is cryopreserved or lyopreserved. A method for preparing an enhanced graft composition with improved osteoinductive properties includes processing demineralized cortical bone; solubilizing growth factors from demineralized bone; and mixing the demineralized cortical bone with the solubilized growth factors.

### CROSS REFERENCE TO RELATED MATTER

This application is a divisional application of U.S. application Ser. No. 18/748,469, filed Jun. 20, 2024, the disclosure of which is hereby incorporated herein in its entirety by this reference.

### FIELD

The present disclosure relates to osteogenic compositions and methods of preparing the same. The osteogenic compositions may contain growth-factor augmented cortical bone and cancellous bone having viable endogenous osteogenic cells that remain adherent to the cancellous bone.

### BACKGROUND

Osteogenic compositions are often used in surgical procedures to rapidly and effectively repair bone. For example, osteogenic compositions can be used to enhance or accelerate the growth of new bone tissue at a surgical site. Osteogenic compositions contain demineralized bone tissue to improve the osteoinductivity needed for growth of new bone tissue. The field of tissue engineering and regenerative medicine has long recognized the pivotal role of growth factors in stimulating tissue repair and regeneration. Growth factors are a group of biologically active proteins that are secreted by cells in response to various stimuli, including injury, stress, or disease. They play a crucial role in modulating cellular activities such as proliferation, differentiation, and migration, which are essential for the healing process. Traditionally, growth factors have been applied to promote tissue healing. This approach has been based on the understanding that these molecules can initiate and support the repair of injured tissue by directly influencing the behavior of cells involved in the healing process.

Demineralized bone matrix (DBM) is a form of allograft using acidic solution to remove mineral components, while leaving much of the proteinaceous components native to bone, with small amounts of calcium-based solids, inorganic phosphates, and some trace cell debris. It is an osteoconductive and osteoinductive biomaterial and is approved as a medical device for use in bone defects and spinal fusion.

Based on known processes for applications of demineralized bone and growth factors, there is a need for innovative approaches that can enhance the osteoinductive properties of biomaterials and improve the efficacy of bone regeneration.

### SUMMARY

The present disclosure relates to osteogenic compositions and methods of preparing the same. In various aspects, an osteogenic composition comprises growth-factor augmented cortical bone and cancellous bone having viable endogenous osteogenic cells that remain adherent to the cancellous bone. The growth-factor augmented cortical bone may include demineralized cortical bone amplified with growth factors derived from a demineralized bone. The cancellous bone may not have been subjected to a loosening agent and the osteogenic composition may be cryopreserved.

In various aspects, an osteogenic composition comprises growth-factor augmented cortical bone and cancellous bone having viable endogenous osteogenic cells that remain adherent to the cancellous bone. The growth-factor augmented cortical bone may include demineralized cortical bone amplified with growth factors derived from a demineralized bone. The cancellous bone may not have been subjected to a loosening agent and the osteogenic composition may be lyopreserved.

The demineralized cortical bone may be milled and/or ground from cortical bone. The demineralized cortical bone may be obtained by milling cortical fibers from a bone, demineralizing the cortical fibers, processing the cortical fibers with an antibiotic solution, and storing the cortical fibers at a low temperature before being mixed with the solubilized growth factors. The demineralized cortical bone may include cortical fibers ranging from about 1 mm to about 2 cm. The demineralized cortical bone may include cortical particulates ranging in size from about 1 μm to about 5 mm.

The demineralized cortical bone may be amplified by exposing the demineralized cortical bone to a solution containing the growth factors. In some embodiments, the growth factors are obtained from demineralized bone (cortical and/or cancellous) that has been soaked in an acidic solution to extract the growth factors. For example, the growth factors may be obtained from demineralized bone that has been contacted with an acidic extraction medium at a pH of between about 0.5 and about 4.4 at an extraction temperature between about 15° C. and 35° C. for between about 24 and 72 hours. The acidic extraction medium may be a citric acid solution. The growth-factor augmented cortical bone may be formed by adding the growth factors to the demineralized bone, and incubating for between about 1 hours and about 12 hours at between about 15° C. and about 44° C. Additionally, and/or alternatively, the method may further include neutralizing the growth factor rich acidic solution with a basic solution, dialyzing the solution before mixing the resulting product with the demineralized cortical bone, and lyophilizing the resulting mixture. Dialyzing the solution may include using a membrane that is between about 6 and about 12 kDa.

The demineralized bone used for extraction may be cortical or cancellous bone. The demineralized bone may include cortical particulates ranging in size from about 1 μm to about 5 mm.

The cancellous bone may be ground.

The method may also include obtaining cancellous bone, grinding the cancellous bone, treating the ground cancellous bone to form a treated cancellous bone, and adding the treated cancellous bone to the lyophilized enhanced graft composition, to form an osteogenic composition. Additionally, the method may include cryoprotecting the osteogenic composition, mixing the solution with the demineralized cortical bone, incubating the mixture, draining the mixture, and rinsing the mixture.

At least two of the demineralized cortical bone, the demineralized bone, and the cancellous bone may be derived from the same bone.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be better understood when read in conjunction with the following drawings wherein like structure is indicated with like reference numerals and in which:

FIG. 1 is a flow diagram illustrating one embodiment for preparing an enhanced graft composition in accordance with the present disclosure.

FIG. 2 is a flow diagram illustrating one embodiment for preparing demineralized cortical bone in accordance with the present disclosure.

FIG. 3 is a flow diagram illustrating one embodiment for preparing a growth factor rich solution from demineralized bone in accordance with the present disclosure.

FIG. 4 is a flow diagram illustrating another embodiment for preparing a growth factor rich solution from demineralized bone in accordance with the present disclosure.

FIG. 5 is a flow diagram illustrating an embodiment for preparing growth-factor augmented cortical bone in accordance with the present disclosure.

FIG. 6 is a flow diagram illustrating another embodiment for preparing growth-factor augmented cortical bone in accordance with the present disclosure.

### DETAILED DESCRIPTION

The present disclosure relates to osteogenic compositions and methods of preparing the same. In one aspect, the osteogenic composition includes: (1) growth-factor augmented cortical bone, the growth-factor augmented cortical bone comprising demineralized cortical bone amplified with growth factors derived from a demineralized bone; and (2) cancellous bone having viable endogenous osteogenic cells that remain adherent to the cancellous bone, the cancellous bone not having been subjected to a loosening agent. The osteogenic composition may be cryopreserved. In other embodiments, the osteogenic composition may be lyopreserved. In other embodiments, the osteogenic composition includes growth-factor augmented cortical bone, the growth-factor augmented cortical bone comprising demineralized cortical bone amplified with growth factors derived from a demineralized bone.

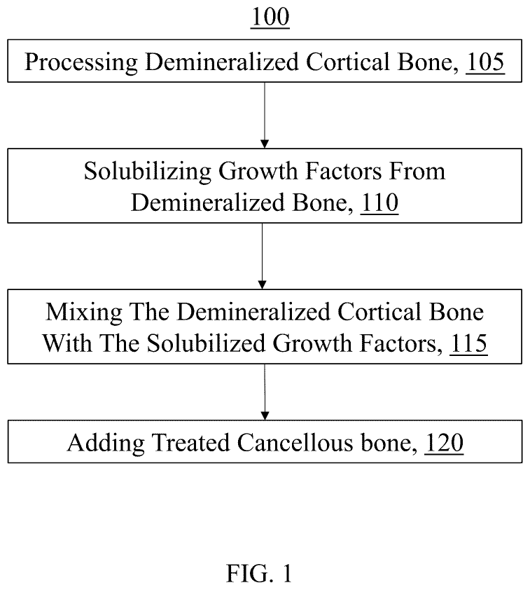

FIG. 1 illustrates a method (100) for preparing an enhanced graft composition with improved osteoinductive properties, and may include (1) processing demineralized cortical bone (105); (3) solubilizing growth factors from demineralized bone (110); and (3) mixing the demineralized cortical bone with the solubilized growth factors (115) to form an enhanced graft composition. In some configurations, an optional act may include adding cancellous bone (120), which may optionally contain cells, to the enhanced graft composition to form an osteogenic composition.

### Cancellous Bone Processing

In some embodiments, the osteogenic composition includes processed cancellous bone. Processing the cancellous bone may include obtaining cancellous bone, grinding the cancellous bone, treating the ground cancellous bone to form a treated cancellous bone, and adding the treated cancellous bone to the lyophilized enhanced graft composition, to form an osteogenic composition.

Any suitable known method can be used to process cancellous bone for the osteogenic compositions described herein. According to one aspect, the cancellous bone is processed such that it has viable endogenous osteogenic cells that remain adherent to the cancellous bone. For example, the cancellous bone in one embodiment is not subjected to a loosening agent. In other embodiments, the cancellous bone may be subjected to a loosening agent. A loosening agent may be an agent that may be contacted with a bone matrix for a time and at a concentration sufficient to loosen osteogenic cells in the cancellous bone matrix without releasing the osteogenic cells from the cancellous bone matrix (i.e., collagenase, a digestive enzyme such as trypsin, amylase, lipase, and combinations thereof).

In one method, cancellous bone is ground. The cancellous bone may be processed by cutting the cancellous bone into pieces small enough to fit in a grinder, and then grinding the cancellous to about 1 mm particle size to form milled cancellous bone. The milled cancellous bone is then washed and subjected to immunodepletion. For example, the milled cancellous bone can be washed with a saline+anticoagulant citrate dextrose solution USP (ACD) Solution A (ACD-A), until solution turns clear. The solution is decanted, and the milled cancellous bone can be rinsed with a mixture of ACD-A in phosphate buffered saline (PBS), and shaken for about 30 seconds. The rinse of ACD-A/PBS can be repeated as necessary (for example, for a total of 2 rinses, or more as needed/desired). In other configurations, other suitable washing steps can be used.

After washing, the washed milled cancellous bone is measured for volume and suitably stored prior to antibiotic treatment. For example, the cancellous bone can be stored in Dulbecco's Minimal Essential Medium (DMEM) at 37° C. prior to antibiotic treatment.

Any suitable antibiotic treatment can be used for the cancellous bone. In one embodiment, the cancellous bone is placed in an antibiotics solution containing gentamycin sulfate, vancomycin HCl and amphotericin B at 37° C. for from about 2 hours to overnight. After antibiotic treatment, the cancellous bone can then be rinsed. In one method, the antibiotic solution is decanted, and PBS is added to the cancellous bone and shaken for about 30 seconds. Then the PBS is decanted, PlasmaLyte-A is added and again the mixture is shaken for about 30 seconds. The PlasmaLyte is decanted, and rinsing with PlasmaLyte-A can be repeated one, two, or more times as desired. Other rinsing methods and/or solutions can also be used.

After the final rinsing, the rinsing solution is decanted, and a cryoprotectant is added and the cancellous bone is stored at about −80° C. in a freezer. Any suitable cryoprotectant may be used and in one embodiment, DMSO-free cryoprotectant is used before storing at −80° C.

### Cortical Bone Processing

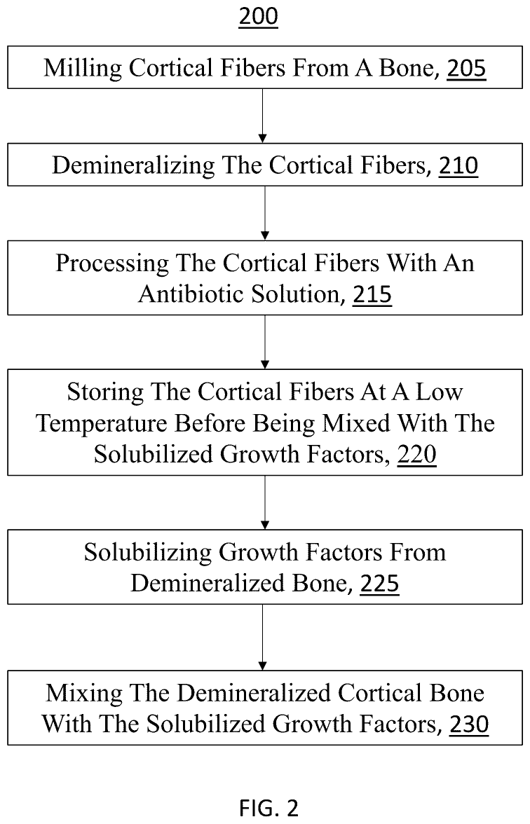

Any suitable known method can be used to process cortical bone for the osteogenic compositions described herein. FIG. 2 illustrates a flow diagram for on embodiment of a method (200) of preparing cortical bone. In this embodiment, the demineralized cortical bone is obtained by milling cortical fibers from a bone (205), demineralizing the cortical fibers (210), processing the cortical fibers with an antibiotic solution (215), and storing the cortical fibers at a low temperature before being mixed with the solubilized growth factors (220) as described below. A method for preparing an enhanced graft may also include solubilizing growth factors from demineralized bone (225); and mixing the demineralized cortical bone with the solubilized growth factors (230).

In one method, the cortical bone is subjected to standard debridement and cleaning procedures. The cortical bone can then be milled/ground as desired. The cortical bone may be milled from bone, ground from bone, etc. Fibers may be milled from the cortical bone using current processing techniques. The cortical fibers may be any suitable size and in one embodiment, the cortical fibers range in size from about 1 mm to about 2 cm. The cortical bone may also comprise cortical particulates, and in one embodiment, the cortical particulates range in size from about 1 μm to about 5 mm. In other embodiments, other types of milling, grinding or grating techniques can be used for other cuts of cortical bone (i.e., non-fibers, powder, particulates, shavings, shards, etc.). The milled cortical bone is demineralized using standard processing techniques.

After demineralization, the milled fibers can be neutralized, such as with PBS, and subjected to antibiotic solution. In one embodiment, milled fibers are neutralized with PBS and soaked in an antibiotic solution for about 2 hours to overnight. After soaking in the antibiotic solution, the antibiotics solution is decanted, and rinsed. For example, PBS can be used to rinse and the PBS is then decanted. The wet milled and demineralized cortical bone (i.e. fibers, non-fibers, powder, particulates, shavings, shards, etc.) can be stored in refrigerated or frozen conditions.

### Growth Factor Processing

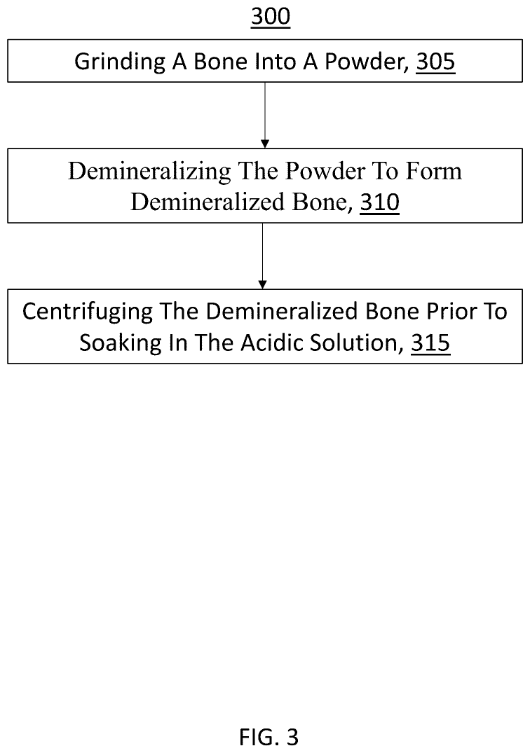

According to the present disclosure, growth factors can be obtained from demineralized bone using an acidic solution soak to extract growth factors. Exposing the demineralized bone to an acidic solution solubilizes the growth factors in the demineralized bone. FIG. 3 illustrates a flow diagram for a method (300) of obtaining growth factors, including grinding a bone into a powder (305) demineralizing the powder to form demineralized bone (310), and centrifuging the demineralized bone prior to soaking in the acidic solution (315).

Any suitable source of demineralized bone can be used. In one embodiment, the source of demineralized bone is from offcuts/stubs leftover from fiber processing and/or other cortical bone that may traditionally go unused. In some configurations, at least two of the demineralized cortical bone, the demineralized bone, and the cancellous bone are derived from the same bone. In one embodiment, demineralized cortical bone and the demineralized bone are derived from the same bone (i.e., the demineralized bone is obtained from the offcut of the cortical bone). In other embodiments, other sources can be used for demineralized bone and different bones are used.

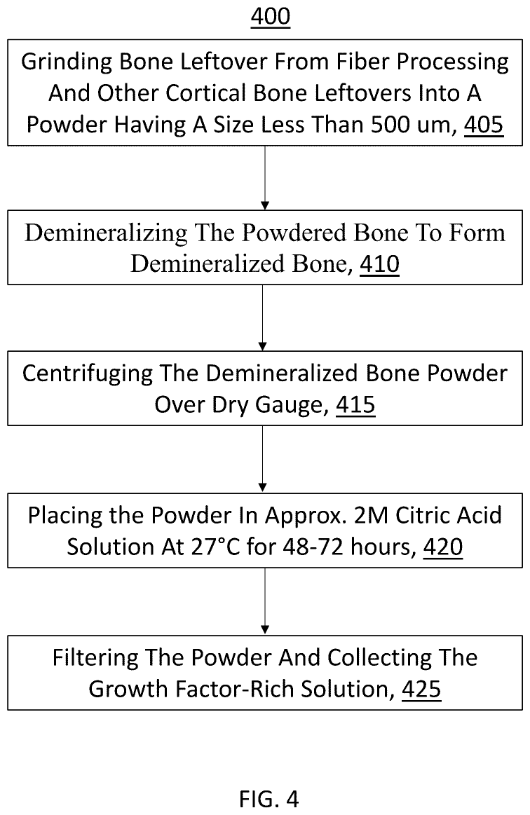

The bone (such as cortical bone from offcuts or any other suitable source of bone) can be ground into powder. FIG. 4 illustrates a flow diagram for a method (400) of obtaining growth factors. In one embodiment, the bone is ground into <500 μm powder (405). The ground bone is then demineralized using standard processing techniques (410). The demineralized ground bone can be centrifuged over dry gauge (415), and then placed in an acidic solution. The demineralized bone may be soaked in an acidic solution to extract growth factors. Growth factors may be obtained from demineralized bone that has been contacted with acidic extraction medium at a pH of between about 0.5 and about 4.4, or from about 0.5 to about 2.0. The extraction temperature may be between about 15° C. and 40° C., or between about 20° C. to 35° C., or between about 25° C. to 30° C., and the time in the acidic solution may be between about 48 and 72 hours. In some embodiments, the acidic extraction medium is an organic acid. Any suitable organic acid may be used (citric acid, acetic acid, etc.). In one embodiment, the demineralized ground bone is placed in 2M Citric Acid Solution at 27° C. for between about 48 and 72 hours (420 in FIG. 4). Other types of acidic solution can also be used as well as other suitable conditions to extract growth factors from the demineralized bone.

After soaking in an acidic solution, the demineralized ground bone is filtered from the solution and the solution (now rich in growth factors from the demineralized ground bone) is collected (425).

### Preparing an Enhanced Graft, or Growth-Factor Augmented Cortical Bone

The growth factor rich solution obtained from the demineralized ground cortical bone can be used to augment cortical bone and form growth-factor augmented cortical bone or enhanced graft. Any suitable method may be used to augment the cortical bone with the growth factor rich solution. The demineralized cortical bone may be amplified or augmented by exposing the demineralized cortical bone to a solution containing growth factors.

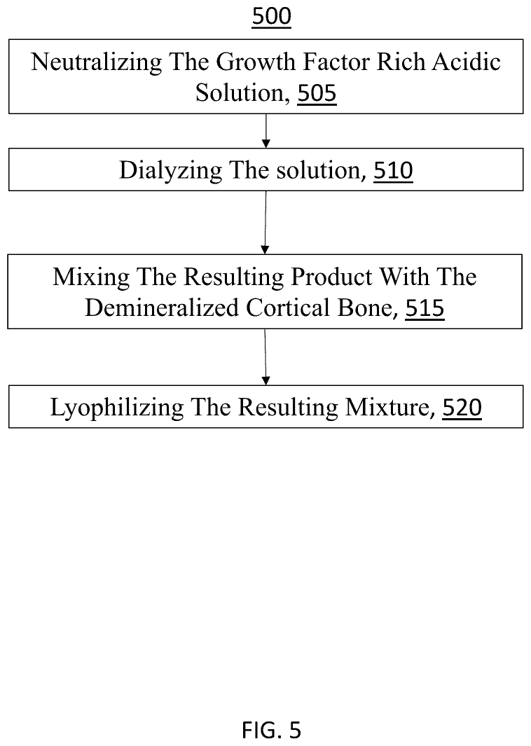

FIG. 5 shows a flow diagram of one method (500) of preparing growth-factor augmented cortical bone: neutralizing the prepared acidic growth factor rich solution (505), dialyzing the solution (510), mixing the resulting product with the demineralized cortical bone (515), and lyophilizing the resulting mixture (520). In one application of this embodiment, a growth-factor augmented cortical bone is formed by neutralizing the growth factors with a basic solution, dialyzing the solution, adding the demineralized milled cortical fibers, and lyophilizing. Any suitable neutralization method may be used. In one embodiment, the growth factor rich solution is neutralized with a basic solution, such as 1M NaOH (1 part solution: 3 parts NaOH). In one embodiment, the solution with 1 part is the extracted solution (this solution is prepared by mixing citric acid with cortical bone powder and then kept for 48-72 hours at 27° C.).

The neutralized solution can be dialyzed. In one embodiment, dialyzing the solution comprises using a membrane that is between about 4 and about 12 kDa, between about 6 and about 10 kDa, or between about 6-8 kDa. In other embodiments, the solution can be dialyzed using membranes with a molecular weight cut-off of 6-8 kDa for about 18 hours. The post-dialysis extract can be mixed with thawed processed cortical bone (such as thawed processed cortical bone fibers processed as described above), and frozen and lyophilized using a standard cycle.

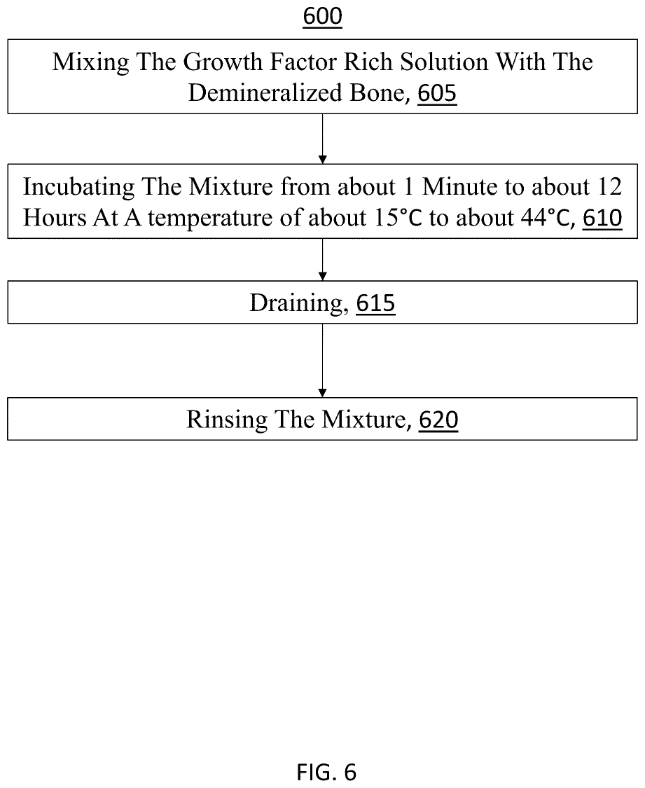

According to another method (600) of preparing an enhanced graft or growth-factor augmented cortical bone as illustrated in the flow diagram of FIG. 6, the prepared growth factor rich solution can be added to demineralized cortical bone (605) (such as thawed processed cortical bone, i.e., thawed processed cortical bone fibers processed as described above), and incubated (610). In one embodiment, growth-factor augmented cortical bone can be formed by mixing the growth factor rich acidic solution with the demineralized cortical bone (605), incubating the mixture (610), draining (615), and rinsing the mixture (620).

In one embodiment, the growth-factor augmented cortical bone is formed by adding the growth factors to the demineralized bone, and incubating for between about 1 minute to about 12 hours (e.g., 5 minutes, 10 minutes, 20 minutes, 30 minutes, 45 minutes, 1.5 hours, 3.5 hours, 4.5 hours, 7 hours, 8 hours, 9 hours, 10 hours, 10.5 hours, 11 hours, or an incubation time within a range defined by any two of the foregoing values), or between about 1 hour and about 3 hours at between about 15° C. and about 44° C., or about 32° C. and about 44° C. In one embodiment, the growth-factor augmented cortical bone is formed by adding the growth factors to the demineralized bone, and incubating for about 2 hours at about 37° C. Other incubation conditions can also be used. The solution can then be neutralized, such as with PBS (2 or more times as needed) and decanted. This osteogenic composition may be cryoprotected, and/or cancellous bone can be added as described below.

### Preparing an Osteogenic Composition

The growth-factor augmented cortical bone or enhanced graft can be combined with the processed cancellous bone to form an osteogenic composition with improved osteo-conductive properties. The processed cancellous bone that was previously processed and frozen as described above can be thawed with DMSO-free cryoprotectant. The thawed cancellous bone can be mixed with the growth-factor augmented cortical bone (prepared according to any of the methods described herein) and cryoprotected. Cancellous bones with or without cells can be used as desired by the surgeon. For example, processed cancellous bone as described above may be lyophilized and therefore not contain cells. In other embodiments, cancellous bone that has been processed according to other known methods may not be lyophilized and contain cells and can be combined with the growth-factor augmented cortical bone or enhanced graft.

In one embodiment, the mixture of cancellous bone and growth-factor augmented cortical bone can be frozen at any suitable temperature, for example at about −80° C. The composition can then be tested for sterility per USP<71> (i.e., a sterility level in accordance with United States Pharmacopeia standard <71>).

### Embodiments

The following embodiments are provided as examples only of specific configurations, materials, arrangements, etc. contemplated by the authors of this disclosure:
Embodiment 1: An osteogenic composition comprising: growth-factor augmented cortical bone, the growth-factor augmented cortical bone comprising demineralized cortical bone amplified with growth factors derived from a demineralized bone; and cancellous bone having viable endogenous osteogenic cells that remain adherent to the cancellous bone, the cancellous bone not having been subjected to a loosening agent; wherein the osteogenic composition is cryopreserved.
Embodiment 2: An osteogenic composition comprising: growth-factor augmented cortical bone, the growth-factor augmented cortical bone comprising demineralized cortical bone amplified with growth factors derived from a demineralized bone; and cancellous bone having viable endogenous osteogenic cells that remain adherent to the cancellous bone, the cancellous bone not having been subjected to a loosening agent; wherein the osteogenic composition is lyopreserved.
Embodiment 3. The osteogenic composition of either embodiment 1 or 2, wherein the demineralized cortical bone is amplified by exposing the demineralized cortical bone to a solution containing the growth factors.
Embodiment 4. The osteogenic composition of any one of embodiments 1 through 3, wherein the demineralized cortical bone is milled from bone.
Embodiment 5. The osteogenic composition of any one of embodiments 1 through 3, wherein the demineralized cortical bone is ground from bone.
Embodiment 6. The osteogenic composition of any one of embodiments 1 through 5, wherein the demineralized bone is an offcut.
Embodiment 7. The osteogenic composition of any one of embodiments 1 through 6, wherein the cancellous bone is milled from bone.
Embodiment 8. The osteogenic composition of any one of embodiments 1 through 7, wherein at least two of the demineralized cortical bone, the demineralized bone, and the cancellous bone are derived from the same bone.
Embodiment 9. The osteogenic composition of any one of embodiments 1 through 8, wherein the growth factors are obtained from demineralized bone that has been soaked in an acidic solution to extract the growth factors.
Embodiment 10. The osteogenic composition of any one of embodiments 1 through 8, wherein the growth factors are obtained from demineralized bone that has been contacted with acidic extraction medium at a pH of between about 0.5 and about 4.4 at an extraction temperature between about 15° C. and 35° C. for between about 48 and 72 hours.
Embodiment 11. The osteogenic composition of embodiment 10, wherein the acidic extraction medium is a citric acid solution.
Embodiment 12. The osteogenic composition of any one of embodiments 1 through 11, wherein the growth-factor augmented cortical bone is formed by adding the growth factors to the demineralized bone, and incubating for between about 1 hours and about 3 hours at between about 32° C. and about 44° C.
Embodiment 13. The osteogenic composition of any one of embodiments 2 through 12, wherein the growth factors are formed by neutralizing the growth factors with a basic solution, dialyzing the solution, adding the demineralized milled cortical fibers, and lyophilizing.
Embodiment 14. A method for preparing an enhanced graft composition with improved osteoinductive properties, the method comprising processing demineralized cortical bone; solubilizing growth factors from demineralized bone; and mixing the demineralized cortical bone with the solubilized growth factors.
Embodiment 15. The method of embodiment 14, wherein the demineralized cortical bone and the demineralized bone are derived from the same bone.
Embodiment 16. The method of either one of embodiments 14 or 15, wherein the demineralized bone from which the growth factors are solubilized is an offcut.
Embodiment 17. The method of any one of embodiments 14 through 16, wherein the demineralized cortical bone comprises cortical fibers.
Embodiment 18. The method of embodiment 17, wherein the cortical fibers range in size from about 1 mm to about 2 cm.
Embodiment 19. The method of any one of embodiments 14 through 18, wherein the demineralized cortical bone is obtained by milling cortical fibers from a bone, demineralizing the cortical fibers, processing the cortical fibers with an antibiotic solution, and storing the cortical fibers at a low temperature before being mixed with the solubilized growth factors.
Embodiment 20. The method of any one of embodiments 14 through 16, wherein the demineralized cortical bone comprises cortical particulates.
Embodiment 21. The method of embodiment 20, wherein the cortical particulates range in size from about 1 μm to about 5 mm.
Embodiment 22. The method of any one of embodiments 14 through 21, wherein solubilizing growth factors comprises exposing the demineralized bone to an acidic solution.
Embodiment 23. The method of embodiment 22, wherein obtaining growth factors comprises grinding a bone into a powder, demineralizing the powder to form demineralized bone; and centrifuging the demineralized bone prior to soaking in the acidic solution.
Embodiment 24. The method of either one of embodiments 22 or 23, wherein the acidic solution comprises an organic acid.
Embodiment 25. The method of embodiment 24, wherein the organic acid is at least one of citric acid and acetic acid.
Embodiment 26. The method of any one of embodiments 22 through 25, wherein the pH of the solution is from about 0.5 to about 2.0.
Embodiment 27. The method of any one of embodiments 22 through 26, wherein the temperature of the solution is from about 25° C. to 30° C.
Embodiment 28. The method of any one of embodiments 22 through 27, wherein the demineralized bone is exposed to the acidic solution for a period of about 24 hours to about 72 hours.
Embodiment 29. The method of any one of embodiments 22 through 28, further comprising neutralizing the acidic solution, dialyzing the solution before mixing the resulting product with the demineralized cortical bone, and lyophilizing the resulting mixture
Embodiment 30. The method of embodiment 29, wherein dialyzing the solution comprises using a membrane that is between about 6 and about 12 kDa.
Embodiment 31. The method of either one of embodiments 29 or 30, further comprising obtaining cancellous bone, grinding the cancellous bone, treating the ground cancellous bone to form a treated cancellous bone, and adding the treated cancellous bone to the lyophilized enhanced graft composition, to form an osteogenic composition.
Embodiment 32. The method of embodiment 31, further comprising cryoprotecting the osteogenic composition.
Embodiment 33. The method of any one of embodiments 22 through 28, further comprising mixing the solution with the demineralized cortical bone, incubating the mixture, draining the mixture, and rinsing the mixture.
Embodiment 34. The method of embodiment 33, wherein the incubating lasts from about 1 minute to about 12 hours at a temperature of between about 15° C. and about 44° C.
Embodiment 35. The method of either one of embodiments 33 or 34, further comprising obtaining cancellous bone, grinding the cancellous bone, treating the ground cancellous bone to form a treated cancellous bone, and adding the treated cancellous bone to the enhanced graft composition to form an osteogenic composition.
Embodiment 36. The method of any one of embodiments 30 or 35, wherein at least two of the demineralized cortical bone, the demineralized bone, and the cancellous bone are derived from the same bone.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular application (e.g., spinal implants); however, other applications are also contemplated.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. In one embodiment, the terms “about” and “approximately” refer to numerical parameters within 10% of the indicated range.

The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Furthermore, if any references have been made to patents and printed publications throughout this disclosure, each of these references and printed publications are individually incorporated herein by reference in their entirety.

In closing, it is to be understood that the embodiments disclosed herein are illustrative of the principles of the present disclosure. Other modifications that may be employed are within the scope of this disclosure. Thus, by way of example, but not of limitation, alternative configurations of the embodiments of the present disclosure may be utilized in accordance with the teachings herein. Accordingly, the present disclosure is not limited to that precisely as shown and described.

We claim:

1. A method for preparing an enhanced graft composition with improved osteoinductive properties, the method comprising:
processing demineralized cortical bone;
solubilizing growth factors from demineralized bone; and
mixing the demineralized cortical bone with the solubilized growth factors.

2. The method of claim 1, wherein the demineralized cortical bone and the demineralized bone are derived from the same bone.

3. The method of claim 1, wherein the demineralized bone from which the growth factors are solubilized is an offcut.

4. The method of claim 1, wherein the demineralized cortical bone comprises cortical fibers.

5. The method of claim 4, wherein the cortical fibers range in size from about 1 mm to about 2 cm.

6. The method of claim 1, wherein the demineralized cortical bone is obtained by milling cortical fibers from a bone, demineralizing the cortical fibers, processing the cortical fibers with an antibiotic solution, and storing the cortical fibers at a low temperature before being mixed with the solubilized growth factors.

7. The method of claim 1, wherein the demineralized cortical bone comprises cortical particulates.

8. The method of claim 7, wherein the cortical particulates range in size from about 1 μm to about 5 mm.

9. The method of claim 1, wherein solubilizing growth factors comprises exposing the demineralized bone to an acidic solution to obtain a growth factor rich acidic solution.

10. The method of claim 9, wherein obtaining growth factors comprises grinding a bone into a powder, demineralizing the powder to form demineralized bone; and centrifuging the demineralized bone prior to soaking in the acidic solution.

11. The method of claim 10, further comprising:
neutralizing the acidic solution,
dialyzing the solution,
mixing the dialyzed solution with the demineralized cortical bone, and
lyophilizing the mixture of dialyzed solution and demineralized cortical bone to form a lyophilized enhanced graft composition.

12. The method of claim 11, wherein dialyzing the solution comprises using a membrane that is between about 6 and about 12 kDa.

13. The method of claim 12, further comprising obtaining cancellous bone, grinding the cancellous bone to form ground cancellous bone, treating the ground cancellous bone to form a treated cancellous bone, and adding the treated cancellous bone to form an osteogenic composition.

14. The method of claim 9, further comprising mixing the growth factor rich acidic solution with the demineralized cortical bone, incubating the mixture, draining the mixture, and rinsing the mixture.

15. The method of claim 14, further comprising obtaining cancellous bone, grinding the cancellous bone to form ground cancellous bone, treating the ground cancellous bone to form a treated cancellous bone, and adding the treated cancellous bone to the enhanced graft composition to form an osteogenic composition.

16. The method of claim 15, wherein at least two of the demineralized cortical bone, the demineralized bone, and the cancellous bone are derived from the same bone.
